(19) United States
(12) Patent Application Publication
(10) Pub. No.: US 2005/0228442 A1
(43) Pub. Date: Oct. 13, 2005

(54) Tissue joining devices capable of delivery of bioactive agents and methods for use thereof

(76) Inventors: Margaret A. Wheatley, Media, PA (US); Ari D. Brooks, Cherry Hill, NJ (US)

Correspondence Address: LICATLA & TYRRELL P.C., 66 E. MAIN STREET, MARLTON, NJ, 08053, US

(21) Appl. No.: 10/512,964

(22) PCT Filed: May 6, 2003
(86) PCT No.: PCT/US03/14059

§ 371 Date: Jun. 2, 2005

Related U.S. Application Data
(60) Provisional application No. 60/378,715, filed on May 6, 2002.

(51) Int. Cl. A61B 17/08; A61D 1/00

(52) U.S. Cl. 606/213; 606/153

(57) ABSTRACT

Tissue joining devices of one or more bending, interconnecting, or magnetically attractive components (1, 2) are provided. Methods for delivering bioactive agents (3) via these tissue joining devices (1, 2) are also provided.

### FIELD OF THE INVENTION

The present invention provides tissue joining devices preferably loaded with a bioactive agent. The tissue joining devices are composed of a biocompatible polymer, ceramic or metal closing means of one or more bending, interconnecting or magnetically attractive components. In a preferred embodiment, the component or components bend, interconnect or magnetically attract so that there is a variable gap size between the components. Also preferred is that one or more components be loaded with a bioactive agent and that the tissue joining device be sized for use in vessels or organs with a diameter of 10 mm or less. In a preferred embodiment, the tissue joining device comprises a bioactive agent selected to promote healing, decrease inflammation and prevent infection at an anastomosis site or other surgical site. The tissue joining device can also be loaded with bioactive agents such as chemotherapeutic agents for local delivery to a surgical site, i.e. following tumor resection, and with bioactive agents such as imaging agents for detection and monitoring of the tissue joining device or surrounding area.

### BACKGROUND OF THE INVENTION

A variety of mechanical devices used for closing of wounds and anastomizing large diameter vessels such as the intestines have been developed over the past century.

Introduction of surgical steel staples to the United States in the 1970's revolutionized the practice of surgery. Because of their reliability, surgical steel staples are now used routinely in many procedures. Three basic designs for staplers exist, the linear stapler, the linear cutting stapler and the circular end to end anastomizing stapler.

Bio-absorbable staples prepared from biodegradable polymeric agents are also commercially available and have been shown to be equivalent but not superior, to steel in safety and efficacy.

U.S. Pat. No. 5,618,313 issued Apr. 8, 1997 and discloses absorbable surgical articles including sutures, clips and other fasteners, staples, pins, screws, prosthetic devices, wound dressings, drug delivery devices, anastomosis rings and other implantable devices formed from copolymerization of dioxanone and other bioabsorbable monomers. Incorporation of medico-surgically useful substances into these articles, such as a substance that accelerates or beneficially modifies the healing process is also disclosed.

U.S. Pat. No. 5,578,662 issued Nov. 26, 1996 and discloses star polymers of soft segment forming monomers useful in forming surgical devices such as sutures, staples, clips, anastomosis rings, bone plates, and screws, and matrices providing for sustained and/or controlled release of pharmaceutically active ingredients. Incorporation of one or more medico-surgically useful substances into the surgical device is also disclosed.

However, size, shape and basic mechanism for attachment of tissues by staples has limited their use, particularly in vessels or organs wherein lumen diameter is 10 mm or less. For example, areas such as blood vessels, bile ducts and ureters have not benefited from the reliability of staples because of their small size and susceptibility to scarring.

### SUMMARY OF THE INVENTION

An object of the present invention is to provide a tissue joining device comprising a biocompatible polymer, ceramic or metal closing means with one or more bending or interconnecting components. In a preferred embodiment, the component of the closing means bends or interconnects so that there is a gap variable in size there between. Also preferred is that the component be loaded with a bioactive agent or agents.

In a preferred embodiment, the tissue joining device is sized to fit within a vessel or organ with a lumen diameter of about 10 mm or less.

Another object of the present invention is to provide a method of joining tissue using a tissue joining device comprising a closing means having one or more bending or interconnecting components wherein the tissue is joined by the bent or interconnecting component or components of the closing means with a gap, variable in size, between the bent component or interconnected components.

Another object of the present invention is to provide a method for delivering one or more bioactive agents to an anastomosis site or other surgical site comprising joining tissue at the anastomosis or other surgical site with a tissue joining device of a biocompatible polymer, ceramic or metal closing means with one or more bending or interconnecting components loaded with bioactive agent. In a preferred embodiment, the anastomosis site or surgical site is in a tissue or organ with a lumen diameter of about 10 mm or less.

Yet another object of the present invention is to provide a method for local delivery of a bioactive agent to a tissue or organ comprising inserting in the organ or tissue a tissue joining device of a biocompatible polymer, ceramic or metal closing means with one or more bending or interconnecting components loaded with a bioactive agent or agents.

Yet another object of the present invention is to provide tissue joining devices and methods for delivering bioactive agents via tissue joining devices that are programmed to lose integrity required for joining the tissue at a different rate from the delivery of a bioactive agent.

Yet another object of the present invention is to provide a tissue joining device comprising one or more bending or interconnecting components wherein at least one of the components is loaded with a bioactive agent that can be imaged.

Yet another object of the present invention is to provide a tissue joining device comprising two or more interconnecting components wherein at least one of the components is attracted to the other component by magnetism.

### BRIEF DESCRIPTION OF THE DRAWINGS

FIG. 1 provides a diagram of one embodiment of the tissue joining device of the present invention. As shown in this Figure, in this embodiment, the closing means of the tissue joining device comprises a rivet with spikes 1 for locking as its first component and a ratchet nut 2 as its second component into which the rivet with spikes is inserted. In this embodiment, the bioactive agent is loaded into the closing means as a disk 3 which fits between the rivet and ratchet nut of the closing means.

### DETAILED DESCRIPTION OF THE INVENTION

In the present invention, tissue joining devices are provided which can be sized for use in vessels and organs wherein lumen diameter is 10 mm or less and which are capable of delivery of bioactive agents at the site of insertion of the device. The capability of these devices to deliver a bioactive agent at the site of insertion is useful in conferring resistance to restenosis in narrow vessels that can occur from scar formation. These devices can also be used to deliver a selected bioactive agent or a combination of bioactive agents to a surgical site or may be used in combination with additional bioactive agents delivered separately to an anastomosis site or other surgical site. The present invention is also useful for treating diseases wherein diseased tissue is removed. For example, these devices can be used to deliver antibiotics to a colonic anastomosis after a resection for diverticulitis. These devices can also be used to deliver anti-inflammatory agents such as amino salicylic acid to the site of anastomosis in inflammatory bowel disease, Crohn's disease or ulcerative colitis. Steroids can be delivered to blood vessels when an autoimmune or inflammatory arterial-obliterative process is treated using these devices. The devices of the present invention are particularly useful in delivering chemotherapeutic agents or radioactive agents to a surgical site, such as in tumor resection, thereby assuring delivery of the treatment to the most common site of local tumor recurrence, the surgical margin. The tissue joining devices can also be loaded with heparin or heparin fragments and used for anastomosis of blood vessels to avoid thrombosis.

In one embodiment, the tissue joining devices of the present invention comprise a closing means with one or more components which bend or interconnect so that there is a gap between the bent component or interconnected components, the size of which is controlled upon insertion. The ability to control the size of the gap between the bent component or interconnected components is of particular importance, as conventional staplers have a small or limited range of gap closures and may not be suitable for thin walled or very thick walled structures. By controlling the gap size between the component or components, it is possible to achieve improved wound healing.

In another embodiment, the tissue joining device comprises a closing means with two or more components that are magnetically attracted to each other. The magnetic attraction of the components of the tissue joining device aids in their placement and alignment.

The joining devices of the present invention preferably further comprise a bioactive agent loaded into one or more of the bending, interconnecting or magnetically attractive components of the closing means. The tissue joining device may comprise a single bioactive agent or multiple bioactive agents. Further, when multiple bioactive agents are loaded onto a device with two or more separate components in the closing means, they may be selected so that when the components are interconnected a synergistic affect or a limiting affect can be obtained. For example, interleukin-2 and a peptide vaccine can be loaded onto different components in a tissue joining device near a tumor, and may effectively boost the immunologic tumor killing. Alternatively, TGF-beta and an adhesion molecule may be loaded onto different components in a tissue joining device to accelerate wound healing whereby the leukocytes will bind to the site due to presence of the adhesion molecules, and the TGF-beta will stimulate them to release other cytokines to promote wound healing. Two components of the coagulation cascade, activated factor X and thrombin can also be loaded onto separate components of a tissue joining device so that coagulation is promoted at the site of the tissue joining device. Additionally, these devices may be used for wound closure without the inclusion of any bioactive agents, chemotherapeutic agents, radioactive agents or other therapeutic agents, solely for their efficient wound closure and tissue joining ability.

The bending component or interconnecting components of the closing means may comprise biocompatible polymers, ceramics, and or metals. Examples of polymers useful in the present invention include, but are not limited to, polylactide, a polyglycolide, a polycaprolactone, a copolymer of polylactide and polyglycolide, a copolymer of lactide and lactone, a polysaccharide, a polyanhydride, a polystyrene, a polyalkylcyanoacrylate, a polyamide, a polyphosphazene, a poly(methylmethacrylate), a polyurethane, a copolymer of methacrylic acid and acrylic acid, a copolymer of hydroxyethylmethacrylate and methylmethacrylate, a polyaminoacid, a polypeptide, and natural and synthetic polysaccharides. Preferred polymers are those which are biocompatible and/or biodegradable. In a preferred embodiment the polymer is polylactic co-glycolic acid (PLGA).

Examples of biocompatible ceramics useful in the present invention include, but are not limited to, zirconia, silicon, and hydroxyapatite.

Examples of biocompatible metals useful in the present invention include, but are not limited to, titanium, steel, and alloys comprising cobalt, chromium, molybdenum, nickel, tungsten, aluminum, and vanadium. Metals useful in the present invention may also comprise magnetic iron and iron oxides.

Preferred polymers, ceramics and metals for use in the present invention are those which can be cast, machined, molded or fabricated in accordance with routine procedures known to those skilled in the art of small parts manufacture. For solvent casting methods, preferred solvents include, but are not limited to, methylene chloride, acetone, acetonitrile, tetrahydrofuran, chloroform, pentane, pentene, methyl ethyl ketone, and combinations thereof.

In a preferred embodiment, the component or components of the closing means of the tissue joining device are selected to provide for a tissue joining device with a programmable lifetime in the body. For example, at least one component of the closing means preferably comprises a bio-absorbable polymer loaded with a bioactive agent that degrades over a selected period of time thereby releasing controlled amounts of the bioactive agent over this time period. In embodiments comprising additional interconnecting or magnetically attracted components, the other component or components may comprise the same polymer, a different polymer, a metal or a ceramic, depending upon the lifetime desired for the device. Degradation of a component may vary from days to years by judicial choice of materials (e.g., polymers with different in vivo degradation rates). The integrity of the tissue joining device can also be programmed based upon selection of the component or components of the closing means. For example, the integrity may be programmed so that the device falls apart or disconnects once the wound has healed but continues to deliver the bioactive agent or vice versa. In a preferred embodiment, the component or components of the closing means maintain their integrity for approximately 14 days and their ability to deliver bioactive agent for up to two months.

For purposes of the present invention, by “loaded” it is meant that the bioactive agent is coated onto one or more of the bending or interconnecting components of the closing means, encapsulated within one or more of the bending, interconnecting, or magnetically attracting components of the closing means, inserted between any of the interconnecting components of the closing means, and/or administered simultaneously with the tissue joining device by incorporation of a reservoir containing bioactive agent into the instrument used to insert the tissue joining device which releases bioactive agent onto the tissue joining device or onto the tissue upon insertion of the tissue joining device. Accordingly, the bioactive agent can be adsorbed on, attached to or encapsulated within part or all of the closing means or the bioactive agent can be adsorbed to, attached to or encapsulated within a separate disc which is held between the bent component or interconnected components of the closing means. Further, more than one bioactive agent can be loaded onto the tissue joining device. As will be understood by one of skill in the art upon reading this disclosure, the amount of bioactive agent delivered can be controlled by the amount of bioactive agent loaded into the closing means, the method by which the bioactive agent is loaded into the closing means, and the material from which the closing means is made. For example, the release profile of a bioactive agent coated onto a metal or ceramic component of the closing means may be faster than the release profile of a bioactive agent encapsulated in a bio-absorbable polymer component. By varying these parameters, delivery capacity of the tissue joining device can range from nanograms to grams over time periods of hours to months. Selection of appropriate amounts and time periods for delivery of a bioactive agent will be dependent upon the bioactive agent to be delivered and the purpose for the tissue joining device. For promoting wound healing at the joined tissue, a preferred tissue joining device will deliver nanogram quantities of a bioactive agent such as TGF-beta, daily, for at least 6 weeks.

Examples of bioactive agents which can be loaded into the closing means include, but are not limited to: antineoplastic and anticancer agents such as azacitidine, cytarabine, fluorouracil, mercaptopurine, methotrexate, thioguanine, bleomycin peptide antibiotics, podophyllin alkaloids such as etoposide, VP-16, teniposide, and VM-26, plant alkaloids such as vincristine, vinblastine and paclitaxel, alkylating agents such as busulfan, cyclophosphamide, mechlorethamine, melphalan, and thiotepa, antibiotics such as dactinomycin, daunorubicin, plicamycin and mitomycin, cisplatin and nitrosoureases such as BCNU, CCNU and methyl-CCNU, anti-VEGF molecules, gene therapy vectors and peptide inhibitors such as MMP-2 and MMP-9, which when localized to tumors prevent tumor growth; inflammatory modulators such as cytokines in the TGF-beta family and cyclooxygenase inhibitors; antibiotics and anti-fungals such as beta-lactams, macrolides, lincosamides, aminoglycosides, tetracyclines, polypeptides, sulfonamides, and fluoroquinolines; wound healing agents such as TGF-β, PDGF, EGF, TGF-α, VEGF, IGF-1, FGFs, angiopoietin, KGF, endothelin, TNF-α, Interleukin-1 or -1β, Interleukin-4, Interleukin-6, Interleukin-8, Interleukin-10, Interleukin-18, SLPi, MCP-1, MIP-1α, MIP-2, IFN-α, IFN-β, and IFN-γ; adhesion molecules such as VCAM-1, ICAM-1, ELAM-1, integrins, selectins, and immunoglobulin superfamily; nucleic acids such as mRNA, DNA, antisense oligonucleotides, plasmids and vectors; vaccines such as DNA and RNA vaccines, peptides, immunostimulatory molecules, and modified bacterial and viral agents; immune regulators such as antibodies, glucocortocoids, immunosuppressants, and anti-idiotype antibodies; endocrine agents such as insulin, thyroid hormone, steroid hormones, androgens, estrogens, and somatostatin; coagulation modulators such as heparin, fractionated or unfractionated, anti-platelet agents, thrombolytic agents, streptokinase, urokinase, and tissue plasminogen activator; vascular tone modulators such as nitric oxide, N-omega-nitro-L-arginine methyl ester, alpha or beta agonists, thrombin and fibrin; and proteoglycans and glycosaminoglycans including hyaluronic acid.

By “bioactive agent” it is also meant to be inclusive of imaging or labeling agents for post-insertion visualization of the tissue joining device or surrounding area. For example, radiopaque markers for visualization by X-ray may be loaded into one or more of the interconnecting components of the closing means. Gas bubbles can also be loaded into one or more of the connecting components for visualization by ultrasound. Radionuclides can be loaded into one or more of the components for visualization using nuclear medicine such as gamma emitters such as ⁹⁹Tc, or ¹²⁵I. In addition, a fluorophore can be loaded into one or more of the connecting components for visualization via fluorescence detection. Further beta emitters such as ¹⁸F as in ¹⁸F-FDG can be loaded for PET scans.

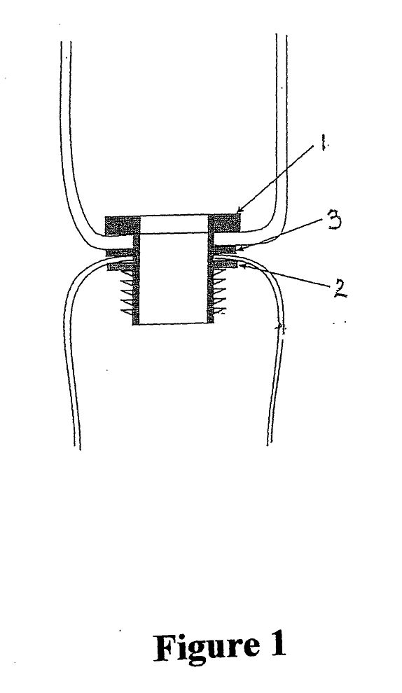

One embodiment of the tissue joining device of the present invention is depicted in FIG. 1. As shown in this Figure, in this embodiment, the closing means of the tissue joining device comprises a rivet with spikes 1 for locking as its first component and a ratchet nut 2 as its second component into which the rivet with spikes is inserted. In this embodiment, the bioactive agent is loaded into the closing means as a disk 3 which fits between the rivet and ratchet nut of the closing means. In this embodiment, the tissue around an open wound is joined by first inserting the first component, the rivet with spikes 1, on one side of the wound and the second component, the ratchet nut 2, on the other side of the wound. The rivet with spikes 1 is then interconnected to the ratchet nut 2 by insertion of the rivet with spikes 1 into the ratchet nut 2 so that tissue on either side of the wound is joined together. An array of small rivets with ratchetable gaps of 1-2 millimeter size can be arrayed for circular, linear, or other alignment. While an open wound has been used for exemplary purposes to explain attachment of this embodiment, as will be understood by one of skill in the art upon reading this disclosure, this embodiment can also be used for all other purposes of the tissue joining devices as described herein. Further, in addition to surgical placement, placement of the tissue joining device may be achieved through other known procedures including, but not limited to, endoscopic, laparoscopic and percutaneous placement.

As will be understood by those of skill in the art upon reading this disclosure, FIG. 1 merely depicts one embodiment of a closing means with two or more interconnecting components. Examples of other closing means with one or more interconnecting components include, but are not limited to: a single component which bends or folds over to interconnects with itself, a single ending a non-circular cross section system, a system in which a first component screws into a second component; a system in which a first component inserts into a second component, and then folds in or out to secure the components in place, i.e. a staple-type mechanism; and a system in which the first component and the second component have spikes which are insertable into each other, i.e. a nut and rivet type mechanism. Alternatively, the device may comprise two or more components magnetically attracted to each other. Design of additional closing means with one or more interconnecting components can also be performed routinely by those of skill in the art based upon the teachings provided herein.

The tissue joining devices of the present invention are advantageous in that they can be rapidly inserted thereby decreasing operation time. In addition, these tissue joining devices can decrease wound healing time through delivery of bioactive agents that promote healing, inhibit inflammation and/or prevent infection directly to a site of anastomosis or other surgery. Further, the tissue joining devices of the present invention can be used to locally treat a surgical site following resection of a disease tissue, i.e. tumor resection, with a chemotherapeutic agent.

The following nonlimiting examples are provided to further illustrate the present invention.

### EXAMPLES

### Example 1

### Fabrication and Testing of Polymeric Compounds for Use in Tissue Joining Devices Loaded with Model Bioactive Agents

Three classes of bioactive agents are evaluated based upon molecule weight and ease of assay for released drug in vitro. 5-Fluorouracil can be used for the small compounds category, insulin can be used for the peptide category, and fluoroscein isothiocyanate labeled bovine serum albumin can be used for the large protein category. Transforming growth factor β3 (TGF-β3) is a 25 kDA protein composed of two disulfide-linked chains of 112 amino acids each. Antibodies to TGF-β(1 and 2) are also available and consist of very high molecular weight IgGs.

Initial studies employed fluoroscein isothiocyanate labeled bovine serum albumin, a fluorescently labeled protein of molecular weight 68,000.00, as a model protein. The molecular weight lies between TGF-β3 and its antibody, and the fluorescent label allows for rapid and quantitative detection.

Materials and Methods:

Polymer, 50:50 PLGA, 85:15 PLGA, PDLLA and PLLA are obtained from BI Chemicals, Henley Division (Montvale, N.J.) and Birmingham Polymers Inc. (Birmingham Ala.) or Alkermes, (Cincinnati, Ohio). All solvents are of Optima grade and obtained from Fisher Scientific (Fair Lawn, N.J.), together with buffer salts. Fluorescently labeled bovine serum albumin (FITC-BSA) was obtained from Molecular Probes (Eugene Oreg.). Other proteins (TGF-β, antibodies), diagnostic kits and radionuclides can be obtained from Sigma Chemical (St. Louis, Mo.), enzyme-linked immunosorbent assays (ELISA) TGF-β3 Quantikine kit can be obtained from R&D Systems (Minneapolis, Minn.).

Prototype Fabrication:

Data was collected using a prototype ring made by solvent casting from methylene chloride.

In solvent casting, a 10-60 wt % solution of polymer in methylene chloride containing 5 to 60% ground, sieved FITC-BSA was cast into a teflon mold and air-dried for one week, then dried under vacuum (9×10⁻⁶ Torr) for one week, until the film maintains constant weight. Rings were carefully removed from the mold when dry.

In precipitation casting, polymer and drug is dissolved and dispersed as above. However, acetone is used as the solvent and then precipitated in ethanol to obtain a tacky precipitate as the solvent that is molded into a cylindrical mold and dried as above.

Release Experiments:

Drug-containing rings of known weight and composition were placed in capped vials containing 4 mL of phosphate buffered saline (PBS), and placed in a 37° C. incubator on a rotary shaker. The buffer was changed daily to mimic the infinite sink conditions of the body, and analyzed for release of entrapped drug. FITC-BSA was measured by fluorescence measurements at excitation and emission wavelengths of 356 nm and 519 nm, respectively. Insulin can be measured by standard immuno-assay. 5-fluorouracil can be assayed by a standard fluorescence assay. The concentration of TGF-βcan be measured using ELISA.

Cumulative releases of FITC-BSA were plotted against time. All assays were performed five times and the results expressed as ±standard deviation. Single factor t analysis of variance was used to determine statistical significance of the results. A two-tailed unpaired t test between sample sets was employed for multiple comparison tests at a significance level of 95%.

The release of BSA and the integrity of the ring were found to correlate significantly with ring composition. For example, comparison of rings made from 50:50 PLGA and pure PLA, revealed release of a therapeutically valuable burst of drug on day one by both rings, with the pure PLA being higher. Very low levels of sustained release were observed in both rings for up to two weeks at which point the PLGA ring produced another burst of release, much larger than the first, and lost integrity.

### Example 2

### Measurement of in vitro Drug Release and Polymer Degradation Profiles Under Physiological Conditions

Degradation Experiments:

Polymer rings were weighed and placed in PBS adjusted to a pH of 7.4 and stored in a 37° C. incubator for the duration of the experiment. The rings were collected every day, lightly dabbed with tissue paper to remove excess buffer, and measured for swelling using a set of calipers. The measured rings were resuspended in fresh buffer and returned to the incubator following measurement. The used buffer was analyzed for breakdown products.

The degradation pattern can be followed by monitoring either changes in molecular weight of the polymer or by tracing the amounts of lactic and glycolic acid released into the solution. Test rings are weighed and placed in PBS adjusted to a pH of 7.4 and stored in a 37° C. incubator for the duration of the experiment. The rings are collected every day for five consecutive days, dissolved in tetrahydrofuran (5 mg/ml) and analyzed via GPC (Hewlett Packard series 1100 with an HP GPC-Addon Rev.A.01.01 column). The elution peaks are monitored by UV absorption at 230 nm. This analysis provides the molecular weight of the remaining polymer, not the soluble breakdown products. Soluble breakdown products released into the incubation buffer are examined via high pressure liquid chromatography (HPLC) using a Waters 2690 Separations Module. The HPLC method used is crucial in separating the lactic and glycolic acids from the buffer solution. The Inertsil ODS-3V, 5 μm column from Alltech separates the two acids based on hydrophobicity. Glycolic acid is much more hydrophilic then lactic acid and will go through the column faster, getting extracted in around 3 minutes as compared to the 5 minutes that lactic acid takes to go through the column. The mobile phase for the separation of the two acids was 0.1M Ammonium Dihydrogen Phosphate, pH 2.5, with a flow rate of 1.0 ml/minute with UV detection at 210 nm. Quantitative data was collected by comparison of elution peak areas with known standards of lactic and glycolic acids

Samples of the rings can also be gold-coated and examined with an Amray Model 1830 scanning electron microscope (Amray, Bedford, Mass.) at 20 kV. Samples are taken before release, and at intervals throughout the release. Prior to gold sputtering, the samples are washed with distilled water, freeze-dried and weighed. Weight loss with time is then plotted.

### Example 3

### Comparison of Tissue Joining Devices Loaded with Bioactive Agent and Application of a Bioactive Agent at the Site of the Tissue Joining Device

Two methods for simultaneous stapling and drug delivery are compared. In the first method, a modified bio-absorbable staple loaded with the bioactive agent is prepared for use in areas such as blood vessels, ureters and bile ducts. In the second method, bioactive agent is administered simultaneously upon insertion of the staple by incorporating a reservoir filled with the bioactive agent into the stapler which is punctured so that bioactive agent is released onto the tissue as the staple is inserted.

1. A tissue joining device comprising a biocompatible polymer, ceramic or metal closing means of one or more bending interconnecting, or magnetically attractive components loaded with a bioactive agent.

2. The tissue joining device of claim 1 wherein at least one of the bending, interconnecting or magnetically attractive components of the closing means comprises a bio-absorbable polymer.

3. The tissue joining device of claim 1 wherein the bioactive agent is loaded as a disk between the bending, interconnecting or magnetically attractive components of the closing means.

4. The tissue joining device of claim 1 which is sized to fit within a vessel or organ with a lumen diameter of about 10 mm or less.

5. The tissue joining device of claim 1 wherein the polymer, ceramic or metal of the closing means is selected based upon its release profile or degradation time to provide for programmed release of the bioactive agent and/or loss of integrity of the tissue joining device.

6. A tissue joining device comprising a closing means of one or more components which bend, interconnect or attract magnetically so that there is a variable gap size between the bent, interconnected or magnetically attracted component or components.

7. The tissue joining device of claim 6 wherein at least one of the components is loaded with a bioactive agent.

8. The tissue joining device of claim 1 or 7 wherein the bioactive agent is an imaging agent.

9. A method for delivering a bioactive agent to an anastomosis site or other surgical site comprising joining tissue at the anastomosis or other surgical site with the tissue joining device of claim 1 or 7.

10. The method of claim 9 further comprising administering an additional bioactive agent directly to the anastomosis site or other surgical site.

11. A method for local delivery of a bioactive agent to a tissue or organ comprising inserting in the organ or tissue a tissue joining device of claim 1 or 7.
